(19) United States
(12) Patent Application Publication
(10) Pub. No.: US 2018/0368417 A1
(43) Pub. Date: Dec. 27, 2018

(54) ANTIMICROBIAL COMPOSITIONS AND METHODS

(71) Applicant: ATTOSTAT, INC., North Salt Lake, UT (US)

(72) Inventor: William Harold Niedermeyer, West Jordan, UT (US)

(21) Appl. No.: 16/012,508

(22) Filed: Jun. 19, 2018

Related U.S. Application Data
(62) Division of application No. 14/861,243, filed on Sep. 22, 2015, now abandoned.
(60) Provisional application No. 62/054,152, filed on Sep. 23, 2014.
(60) Provisional application No. 62/054,154, filed on Sep. 23, 2014.

(51) Int. Cl. A01N 59/16; A61K 33/24; A01N 25/04; A01N 25/34

(52) U.S. Cl. CPC A01N 59/16; A61K 33/24; A01N 25/34; A01N 25/04

(57) ABSTRACT

Antimicrobial compositions for killing or deactivating microbes, such as viruses, bacteria, or fungi, include metal nanoparticles, a carrier, and a plurality of metal nanoparticles. The nanoparticles can be selected to have a particle size and particle size distribution to selectively and preferentially kill one of a virus, a bacterium, or a fungus. Antiviral compositions can include nanoparticles having a particle size of 8 nm or less, 1-7 nm, 2-6.5 nm, or 3-6 nm (or up to 10 nm for Ebola virus). Antibacterial compositions can include nanoparticles having a particle size of 3-14 nm, 5-13 nm, 7-12 nm, or 8-10 nm. Antifungal compositions can include nanoparticles having a particle size of 9-20 nm, 10-18 nm, 11-16 nm, or 12-15 nm. Exemplary methods of killing or deactivating microbes include: (1) applying an antimicrobial composition to a substrate containing microbes, and (2) the antimicrobial composition killing or deactivating the microbes.

### CROSS-REFERENCE TO RELATED APPLICATIONS

This application is a division of U.S. patent application Ser. No. 14/861,243, filed Sep. 22, 2015, which claims the benefit of U.S. Provisional Patent Application No. 62/054,152, filed Sep. 23, 2014, and U.S. Provisional Patent Application No. 62/054,154, filed Sep. 23, 2014. The disclosures of the foregoing patent applications are incorporated herein by reference in their entirety.

### BACKGROUND

### 1. Field of the Invention

Disclosed herein are antimicrobial compositions and methods for killing microbes using such compositions.

### 2. Relevant Technology

Diseases old and new caused by microbes such as bacteria, viruses, and fungi continue to plague humans, animals, and plants. In addition to infecting humans, sometimes as epidemics or pandemics, microbial diseases that affect animals and plants raised for food have and can continue to devastate food supplies. Food products are also prone to spoilage as a result of microbes, particularly bacteria and fungi.

Although modern science has yielded many new antimicrobial compositions, some, such as antibiotics, are not always effective because microbes can build up tolerance or immunity to such compositions. In addition, apart from natural immunity and acquired immunity from vaccinations, there are no compositions that can reliably target and selectively destroy viruses.

Besides specific compositions and drugs, there are non-specific ways to combat microbes and microbial diseases, such as burning, harsh chemicals, and partial or complete removal of diseased human, animal, or plant tissues. Because such measures are non-specific and often kill or severely affect the organism being treated, they are usually used as a last resort.

Among the deadliest human diseases are anthrax, caused by Bacillus Anthracis; ebola virsus disease; hemorrhagic fever, caused by Marburg virus; hantavirus; rabies; smallpox; Crimean-Congo hemorrhagic, fever caused by a tick-borne virus; avian influenza (bird flu); severe acute respiratory syndrome (SARS), caused by coronavirus; malaria, caused by mosquitos infected with Plasmodium parasites; typhoid fever caused by Salmanella Typhi bacterium; cholera, an acute intestinal infection caused by Vibrio cholera bacterium; yellow fever, a hemorrhagic fever transmitted by infected mosquitoes; acquired immune deficiency syndrome (AIDS); bubonic plague, caused by Yersinia pestis bacterium; and wound and flesh infections caused by many different bacteria, particularly those which are antibiotic-resistant.

There are also many plant diseases of many types that could threaten the world's food supply. An example of a potentially devastating plant disease is citrus greening disease, which affects citrus trees and for which there is no cure. Citrus greening disease is caused by motile bacteria, Candidatus Liberibacter spp. and transmitted by insects (e.g., various types of psyllids). In the heyday of Florida citrus, around 1970, the number of acres with orange, grapefruit, and specialty fruit orchards surpassed 900,000. Today, it is reportedly slightly more than 500,000 acres as a result of citrus greening disease.

Botulism from consuming spoiled food is a relatively rare but serious and potentially fatal paralytic illness caused by a nerve toxin that is produced by the bacterium Clostridium botulinum.

Among the deadliest viral diseases is Ebola disease, with a mortality rate that is reportedly as high as 90%. Ebola virus disease (EVD), Ebola hemorrhagic fever (EHF), or simply Ebola is a disease of humans and other primates caused by an Ebola virus. Symptoms of the disease can start two days to three weeks after contracting the virus, with a fever, sore throat, muscle pain and headaches. Typically, vomiting, diarrhea and rash follow, along with decreased functioning of the liver and kidneys. Around this time, affected people may begin to bleed both within the body and externally.

EVD is caused by four of five viruses classified in the genus Ebolavirus, family Filoviridae, order Mononegavirales. The four disease-causing viruses are Bundibugyo virus (BDBV), Sudan virus (SUDV), Taï Forest virus (TAFV), and one called simply, Ebola virus (EBOV, formerly Zaire Ebola virus). Ebola virus is the sole member of the Zaire ebolavirus species, and the most dangerous of the known Ebola disease-causing viruses, as well as being responsible for the largest number of outbreaks. The fifth virus, Reston virus (RESTV), is not thought to be disease-causing in humans. The five Ebola viruses are closely related to Marburg viruses.

No specific treatment for EVD is yet available. Efforts to help those who are infected are supportive and include giving either oral rehydration therapy or intravenous fluids. EVD has a high risk of death, killing between 50% and 90% of those infected. EVD was first identified in Sudan (now South Sudan) and the Democratic Republic of the Congo. Efforts are under way to develop a vaccine; however, none yet exists.

Accordingly, there has been and remains a need to find compositions that can reliably target and preferentially kill or deactivate disease-causing microbes without killing or causing undue harm to the organism being treated (e.g., human, animal, or plant).

### SUMMARY

Disclosed herein are antimicrobial compositions and methods for killing or deactivating a wide variety of harmful microbes, such as viruses, bacteria, and fungi, which can infect humans, animals, plants, or food supplies. Also disclosed are methods for making antimicrobial compositions. Unexpectedly, it has now been found that by selecting metal nanoparticles of a particular size and particle size distribution it is possible to target and preferentially kill or deactivate a specific type of microbe.

In some embodiments, an antimicrobial composition may comprise (1) a carrier and (2) a plurality of metal nanoparticles having a particle size and a particle size distribution selected so as to selectively and preferentially kill one of a virus, bacterium, or fungus.

In some embodiments, anti-viral compositions can include metal nanoparticles having a particle size of about 8 nm or less, or about 1 nm to about 7 nm, or about 2 nm to about 6.5 nm, or about 3 nm to about 6 nm. Within these size ranges it is possible to select “designer anti-viral particles” of specific size that are particularly effective in targeting a specific virus.

In some embodiments, an anti-viral composition may comprise a plurality of non-ionic metal nanoparticles having a particle size in a range of about 0.22 nm to about 2 nm, or about 0.22 nm to about 1.5 nm, or about 0.24 nm to about 1 nm, or about 0.27 nm to about 0.7 nm, or about 0.3 nm to about 0.5 nm, or about 0.35 nm to about 0.45 nm. For example, such embodiments may be useful for targeting and preferentially deactivating Ebola viruses.

In some embodiments, anti-viral compositions can include metal nanoparticles having a particle size of about 2 nm to about 10 nm, or about 4 nm to about 10 nm, or about 6 nm to about 10 nm. For example, such embodiments with particles that are 6 nm, 10 nm, or both may be useful for targeting and preferentially deactivating Ebola viruses by associating with, binding to, and/or disrupting the 3CSY glycoprotein found on the surface of Ebola viruses, thereby preventing binding of the virus to cell surface receptors.

In some embodiments, anti-bacterial compositions can include metal nanoparticles having a particle size of about 3 nm to about 14 nm, or about 5 nm to about 13 nm, or about 7 nm to about 12 nm, or about 8 nm to about 10 nm. Within these size ranges it is possible to select “designer anti-bacterial particles” of specific size that are particularly effective in targeting a specific bacterium.

In some embodiments, anti-fungal compositions can include metal nanoparticles having a particle size of about 9 nm to about 20 nm, or about 10 nm to about 18 nm, or about 11 nm to about 16 nm, or about 12 nm to about 15 nm. Within these size ranges it is possible to select “designer anti-fungal particles” of specific size that are particularly effective in targeting a specific fungus.

In some embodiments, metal nanoparticles can comprise spherical-shaped metal nanoparticles having a mean diameter and a particle size distribution wherein at least 99% of the metal nanoparticles have a particle size within 30% of the mean diameter, or within 20% of the mean diameter, or within 10% of the mean diameter and/or wherein at least 99% of the spherical-shaped nanoparticles have a diameter within ±3 nm of the mean diameter, or within ±2 nm of the mean diameter, or within ±1 nm of the mean diameter.

In some embodiments, metal nanoparticles can comprise coral-shaped metal nanoparticles having a non-uniform cross section and a globular structure formed by multiple, non-linear strands joined together without right angles. In some cases the coral-shaped metal nanoparticles can be used together with spherical-shaped metal nanoparticles (e.g., in order to potentiate the spherical-shaped metal nanoparticles).

In some embodiments, metal nanoparticles can comprises at least one metal selected from the group consisting of gold, platinum, silver, palladium, rhodium, osmium, ruthenium, rhodium, rhenium, molybdenum, copper, iron, nickel, tin, beryllium, cobalt, antimony, chromium, manganese, zirconium, tin, zinc, tungsten, titanium, vanadium, lanthanum, cerium, heterogeneous mixtures thereof, and alloys thereof. Nanoparticles comprised of silver, gold, and mixtures and alloys thereof can be particularly effective.

In some embodiments, a method of killing or deactivating a microbe comprises: (1) applying an antimicrobial composition comprising a carrier and metal nanoparticles onto or into a substrate containing microbes, and (2) the antimicrobial composition killing or deactivating the microbes. The substrate can be a living organism or a non-living object.

In some embodiments, a method of deactivating a virus comprises: (1) applying an anti-viral composition comprising metal nanoparticles having a particle size of about 8 nm or less, or about 1 nm to about 7 nm, or about 2 nm to about 6.5 nm, or about 3 nm to about 6 nm onto or into a substrate containing a virus, and (2) the anti-viral composition deactivating the virus.

In some embodiments, a method of deactivating Ebola viruses comprises: (1) applying an anti-viral composition comprising a carrier and metal nanoparticles onto aa or into an animal or non-living substrate contaminated with Ebola viruses, and (2) the metal nanoparticles deactivating Ebola viruses by attaching to glycoproteins and/or denaturing one or more proteins in the Ebola viruses.

In some embodiments, a method of treating or preventing Ebola virus disease comprises: (1) administering a pharmaceutically acceptable quantity of spherical-shaped nonionic metal nanoparticles having a particle size in a range of about 0.22 nm to about 2 nm to a living organism; and (2) the spherical-shaped nonionic metal nanoparticles deactivating Ebola viruses on or in the living organism.

In some embodiments, a method of treating or preventing Ebola virus disease comprises: (1) administering a pharmaceutically acceptable quantity of spherical-shaped nonionic metal nanoparticles having a particle size in a range of about 2 nm to about 10 nm to a living organism; and (2) the spherical-shaped nonionic metal nanoparticles deactivating Ebola viruses on or in the living organism by binding to, associating with, and/or disrupting the 3CSY glycoproteins of the Ebola viruses.

In some embodiments, a method of killing a bacterium comprises: (1) applying an anti-bacterial composition comprising metal nanoparticles having a particle size of about 8 nm or less, or about 3 nm to about 14 nm, or about 5 nm to about 13 nm, or about 7 nm to about 12 nm, or about 8 nm to about 10 nm onto or into a substrate containing a bacterium, and (2) the anti-bacterial composition killing the bacterium.

In some embodiments, a method of killing a fungus comprises: (1) applying an anti-fungal composition comprising metal nanoparticles having a particle size of about 9 nm to about 20 nm, or about 10 nm to about 18 nm, or about 11 nm to about 16 nm, or about 12 nm to about 15 nm onto or into a substrate containing a fungus, and (2) the anti-fungal composition killing the fungus.

These and other advantages and features of the invention will be set forth in part in the description which follows, and in part will become apparent to those skilled in the art upon examination of the following or may be learned by practice of the invention.

### BRIEF DESCRIPTION OF THE DRAWINGS

FIG. 1 is a transmission electron microscope image (TEM) of exemplary spherical-shaped metal nanoparticles having substantially uniform size and narrow particle size distribution for use in making antimicrobial compositions;

FIGS. 2A-2E are transmission electron microscope images (TEMs) of exemplary coral-shaped metal nanoparticles for use in making antimicrobial compositions;

FIG. 3 schematically illustrates a microbe after having absorbed spherical-shaped metal nanoparticles from a substrate;

FIG. 4 schematically illustrates a microbe protein with disulfide bonds being catalytically denatured by an adjacent spherical-shaped nanoparticle;

FIG. 5 schematically illustrates a mammalian protein with disulfide bonds that are shielded so as to resist being catalytically denatured by an adjacent spherical-shaped nanoparticle; and

FIG. 6 is a scanning electron microscope image (SEM) of an Ebola virus.

### DETAILED DESCRIPTION OF THE PREFERRED EMBODIMENTS

Disclosed herein are antimicrobial compositions and methods for killing or deactivating microbes, such as viruses (including Ebola viruses), bacteria, or fungi. In some embodiments, metal nanoparticles are dispersed within or contained on or within a carrier that can be applied onto or into a substrate containing a microbe. The carrier can be a liquid, gel or solid. The antimicrobial compositions can be formulated to selectively and preferentially kill one or more specific microbes.

In some embodiments, antimicrobial compositions may comprise a carrier and a plurality of metal nanoparticles having a particle size and a particle size distribution selected so as to selectively and preferentially kill one of a virus, a bacterium, or a fungus.

In some embodiments, anti-viral compositions comprise metal nanoparticles having a particle size of about 8 nm or less, or about 0.22 nm to about 2 nm, or about 0.22 nm to about 1.5 nm, or about 0.24 nm to about 1 nm, or about 0.27 nm to about 0.7 nm, or about 0.3 nm to about 0.5 nm, or about 0.35 nm to about 0.45 nm, or about 1 nm to about 7 nm, or about 2 nm to about 6.5 nm, or about 3 nm to about 6 nm, or about 2 nm to about 10 nm, or about 4 nm to about 10 nm, or about 6 nm to about 10 nm.

In some embodiments, anti-bacterial compositions can include metal nanoparticles having a particle size of about about 3 nm to about 14 nm, or about 5 nm to about 13 nm, or about 7 nm to about 12 nm, or about 8 nm to about 10 nm.

In some embodiments, anti-fungal compositions can include metal nanoparticles having a particle size of about 9 nm to about 20 nm, or about 10 nm to about 18 nm, or about 11 nm to about 16 nm, or about 12 nm to about 15 nm.

Within any of the foregoing size ranges, it is possible to select “designer antimicrobial particles” of specific size that are particularly effective in targeting a specific microbe.

The ability to select and use microbe-specific nanoparticles provides a number of benefits. In the case where only certain nanoparticle sizes are effective in killing a particular microbe or class of microbes, providing metal nanoparticles within a narrow particle size distribution of the correct particle size maximizes the proportion of nanoparticles that are effective in killing the target microbe and minimizes the proportion of nanoparticles that are less effective, or ineffective, in killing the target microbe. This, in turn, greatly reduces the overall amount or concentration of nanoparticles required to provide a desired kill- or deactivation rate of a targeted microbe. Eliminating improperly-sized nanoparticles also reduces the tendency of the composition to kill or harm non-targeted microbes or other cells, such as healthy mammalian or human cells. In this way, highly specific antimicrobial compositions can better target a harmful microbe while being less harmful or even non-toxic to humans, animals, and plants.

### Nanoparticle Configurations

In some embodiments, the metal nanoparticles may comprise or consist essentially of nonionic, ground state metal nanoparticles. Examples include spherical-shaped metal nanoparticles, coral-shaped metal nanoparticles, or a blend of spherical-shaped metal nanoparticles and coral-shaped metal nanoparticles.

In some embodiments, nonionic metal nanoparticles useful for making antimicrobial compositions comprise spherical nanoparticles, preferably spherical-shaped metal nanoparticles having a solid core. The term “spherical-shaped metal nanoparticles” refers to nanoparticles that are made from one or more metals, preferably nonionic, ground state metals, having only internal bond angles and no external edges or bond angles. In this way, the spherical nanoparticles are highly resistant to ionization, highly stable, and highly resistance to agglomeration. Such nanoparticles can exhibit a high ξ-potential, which permits the spherical nanoparticles to remain dispersed within a polar solvent without a surfactant, which is a surprising and unexpected result.

In general, spherical-shaped metal nanoparticles can have a diameter of about 40 nm or less, about 35 nm or less, about 30 nm or less, about 25 nm or less, about 20 nm or less, about 15 nm or less, about 10 nm or less, about 7.5 nm or less, or about 5 nm or less. Nevertheless, selecting spherical-shaped nanoparticles of specific particle size and narrow particle size distribution can be particularly useful for selectively and preferentially killing or deactivating a particular type of microbe, such as a virus, bacterium, or fungus.

In some embodiments, spherical-shaped nanoparticles can have a particle size distribution such that at least 99% of the nanoparticles have a diameter within 30% of the mean diameter of the nanoparticles, or within 20% of the mean diameter, or within 10% of the mean diameter. In some embodiments, spherical-shaped nanoparticles can have a mean particle size and at least 99% of the nanoparticles have a particle size that is within ±3 nm of the mean diameter, ±2 nm of the mean diameter, or ±1 nm of the mean diameter. In some embodiments, spherical-shaped nanoparticles can have a ξ-potential of at least 10 mV, preferably at least about 15 mV, more preferably at least about 20 mV, even more preferably at least about 25 mV, and most preferably at least about 30 mV.

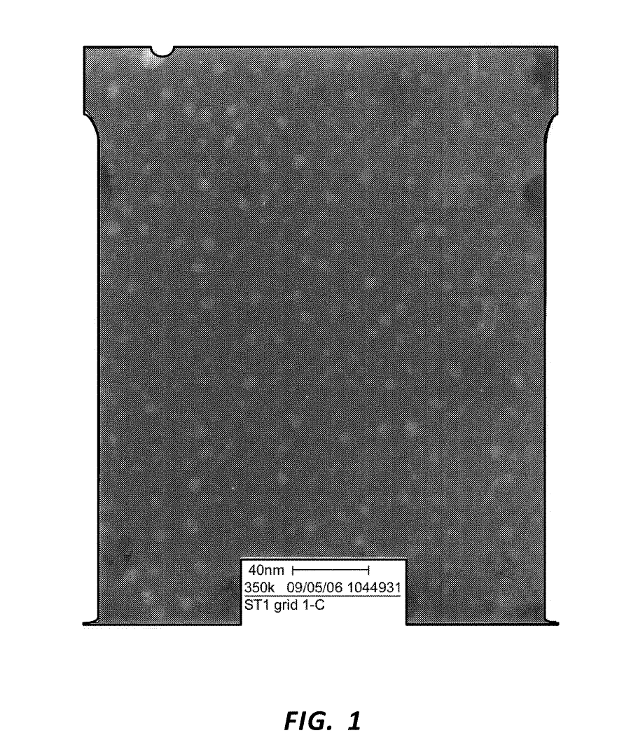

Examples of methods and systems for manufacturing spherical-shaped nanoparticles are disclosed in U.S. Pat. No. 9,849,512 to William Niedermeyer, incorporated herein by reference. FIG. 1 is a transmission electron microscope image (TEM) of exemplary spherical-shaped nanoparticles made using the methods and systems of the Niedermeyer Publication. The illustrated nanoparticles are spherical-shaped silver (Ag) nanoparticles of substantially uniform size, with a mean diameter of about 10 nm and a narrow particle size distribution. In some embodiments, spherical-shaped nanoparticles can have a solid core rather than being hollow, as is the case with conventional metal nanoparticles, which are usually formed on the surfaces of non-metallic seed nanoparticles (e.g., silica), which are thereafter removed to yield hollow nanospheres.

In some embodiments, nonionic metal nanoparticles useful for making antimicrobial compositions may also comprise coral-shaped nanoparticles. The term “coral-shaped metal nanoparticles” refers to nanoparticles that are made from one or more metals, preferably nonionic, ground state metals having a non-uniform cross section and a globular structure formed by multiple, non-linear strands joined together without right angles. Similar to spherical-shaped nanoparticles, coral-shaped nanoparticles may have only internal bond angles and no external edges or bond angles. In this way, coral-shaped nanoparticles can be highly resistant to ionization, highly stable, and highly resistance to agglomeration. Such coral-shaped nanoparticles can exhibit a high ξ-potential, which permits the coral-shaped nanoparticles to remain dispersed within a polar solvent without a surfactant, which is a surprising and unexpected result.

In some embodiments, coral-shaped nanoparticles can have lengths ranging from about 15 nm to about 100 nm, or about 25 nm to about 95 nm, or about 40 nm to about 90 nm, or about 60 nm to about 85 nm, or about 70 nm to about 80 nm. In some embodiments, coral-shaped nanoparticles can have a particle size distribution such that at least 99% of the nanoparticles have a length within 30% of the mean length, or within 20% of the mean length, or within 10% of the mean length. In some embodiments, coral-shaped nanoparticles can have a ξ-potential of at least 10 mV, preferably at least about 15 mV, more preferably at least about 20 mV, even more preferably at least about 25 mV, and most preferably at least about 30 mV.

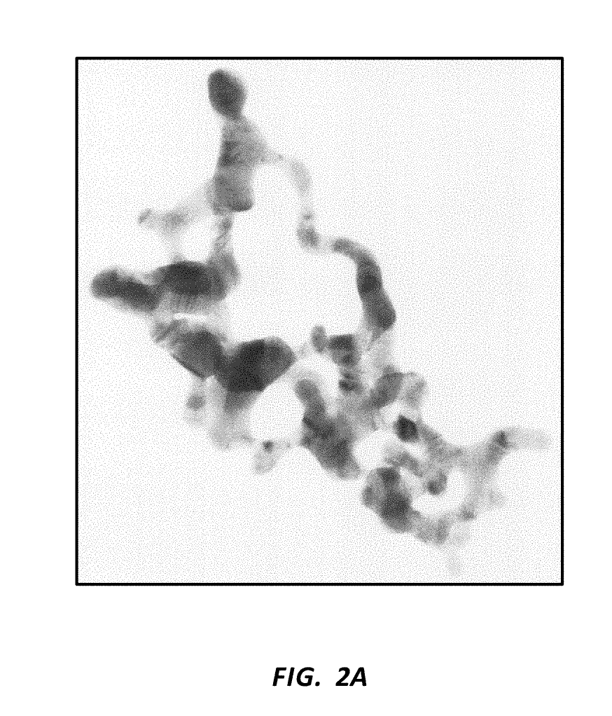

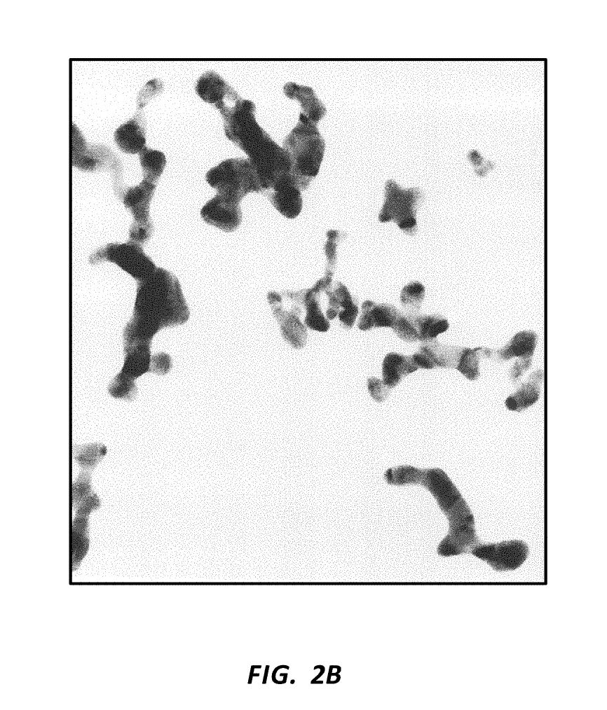

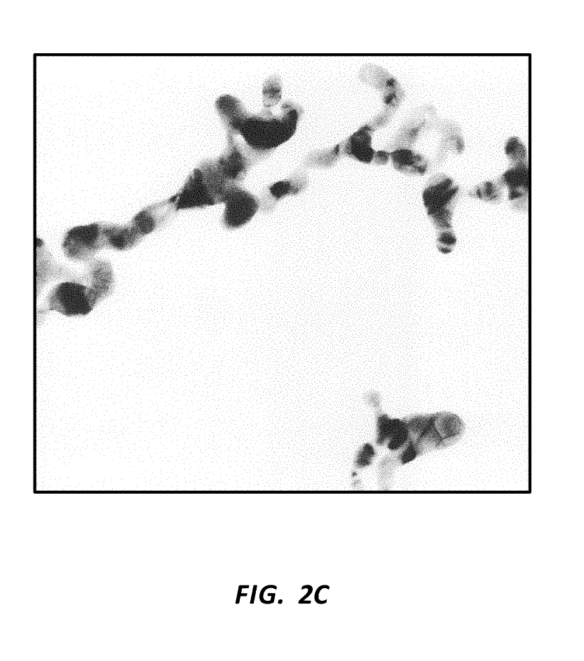

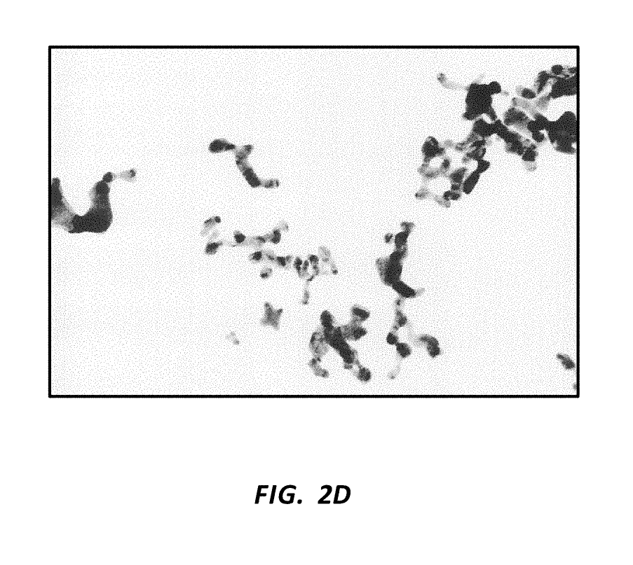

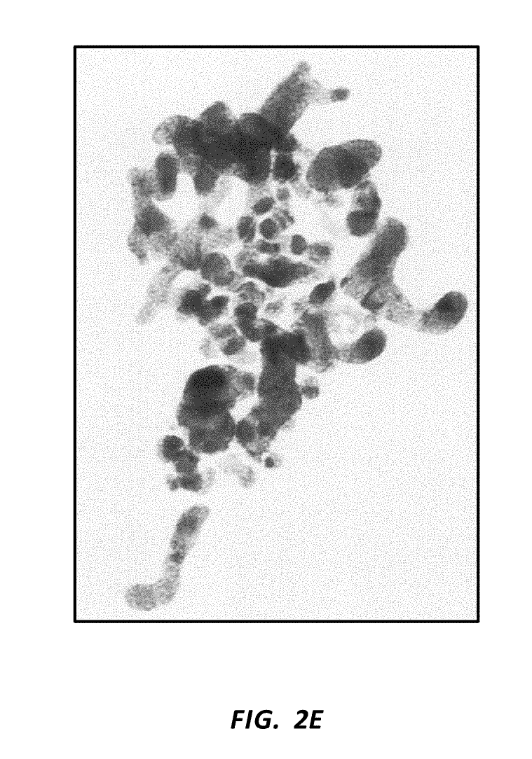

Examples of methods and systems for manufacturing coral-shaped nanoparticles are disclosed in U.S. Pat. No. 9,919,363 to William Niedermeyer, which is incorporated by reference. FIGS. 2A-2E are transmission electron microscope images (TEMs) of exemplary coral-shaped metal nanoparticles made using the methods and systems of the Niedermeyer '369 patent. The illustrated nanoparticles are coral-shaped gold nanoparticles.

The metal nanoparticles, including spherical-shaped and coral-shaped nanoparticles, may comprise any desired metal, mixture of metals, or metal alloy, including at least one of silver, gold, platinum, palladium, rhodium, osmium, ruthenium, rhodium, rhenium, molybdenum, copper, iron, nickel, tin, beryllium, cobalt, antimony, chromium, manganese, zirconium, tin, zinc, tungsten, titanium, vanadium, lanthanum, cerium, heterogeneous mixtures thereof, or alloys thereof.

According to some embodiments, the antimicrobial metal nanoparticles will comprise at least one of silver or gold. In the case of larger metal nanoparticles for use in killing bacteria and fungi, metal nanoparticles may primarily or exclusively comprise silver. However, in the case of smaller metal nanoparticles for use in killing viruses, including Ebola viruses, metal nanoparticles may primarily or exclusively comprise gold. Due to the nature silver and gold atoms making up the nanoparticles, it has been found that gold nanoparticles are better able to hold together at very small sizes (e.g., smaller than about 5-7 nm) compared to silver nanoparticles. On the other hand, in some embodiments, a gold-silver alloy provides the particle stabilizing activity of gold and potentially higher anti-viral activity of silver.

### Antimicrobial Activity

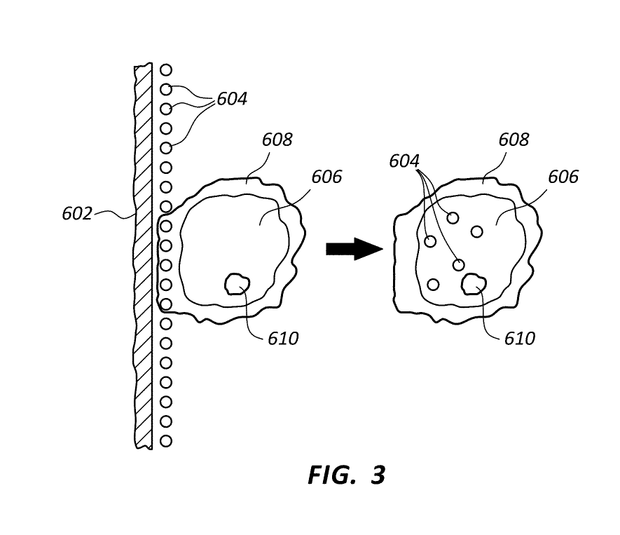

FIG. 3 schematically illustrates a microbe 608 having absorbed spherical-shaped nanoparticles 604 from a solid substrate 602, such as by active absorption or other transport mechanism. Alternatively, spherical-shaped nanoparticles 604 can be provided in a composition (not shown), such as in a liquid or gel carrier. The nanoparticles 604 can freely move throughout the interior 606 of microbe 608 and come into contact with one or more vital proteins or enzymes 610 that, if denatured, will kill or disable the microbe.

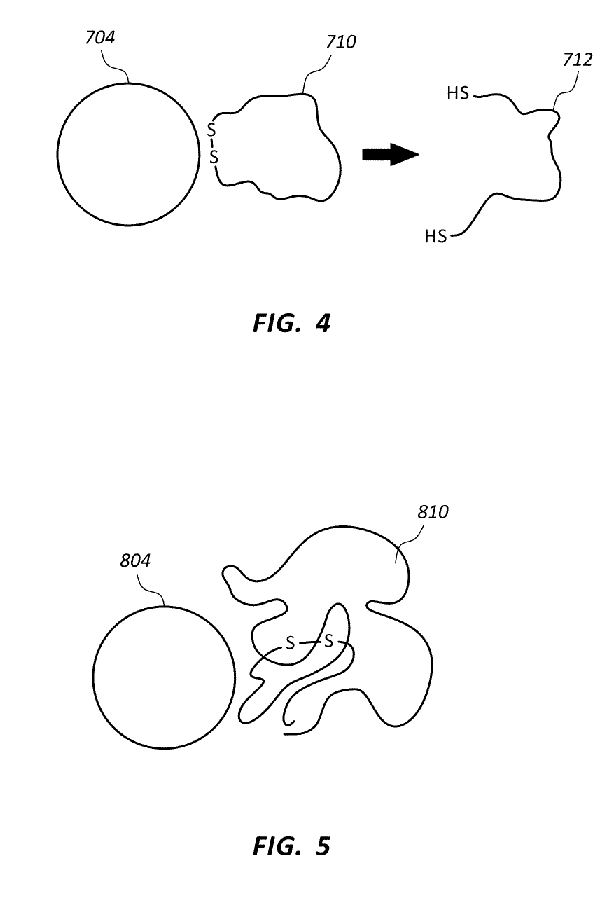

One way that nanoparticles may kill or denature a microbe is by catalyzing the cleavage of disulfide (S—S) bonds within a vital protein or enzyme. FIG. 4 schematically illustrates a microbe protein or enzyme 710 with disulfide bonds being catalytically denatured by an adjacent spherical-shaped nanoparticle 704 to yield denatured protein or enzyme 712. In the case of bacteria or fungi, the cleavage of disulfide bonds and/or cleavage of other chemical bonds of vital proteins or enzymes may occur within the cell interior and thereby killing the microbe in this manner. Such catalytic cleavage of disulfide (S—S) bonds is facilitated by the generally simple protein structures of microbes, in which many vital disulfide bonds are on exposed and readily cleaved by catalysis.

Another mechanism by which metal (e.g., silver) nanoparticles can kill microbes is through the production of active oxygen species, such as peroxides, which can oxidatively cleave protein bonds, including but not limited to amide bonds.

In the case of viruses, spherical-shaped and coral-shaped metal nanoparticles can alternatively deactivate viruses by attaching to glycoproteins and/or catalyzing protein denaturing reactions in the protein coat so that the virus is no longer able to attach to a host cell and/or inject genetic material into the host cell. Because very small nanoparticles can pass through a virus, denaturing of the protein coat may occur within the interior of the virus. A virus that is rendered unable to attach to a host cell and/or inject genetic material into the host cell is essentially inactive and no longer pathogenic.

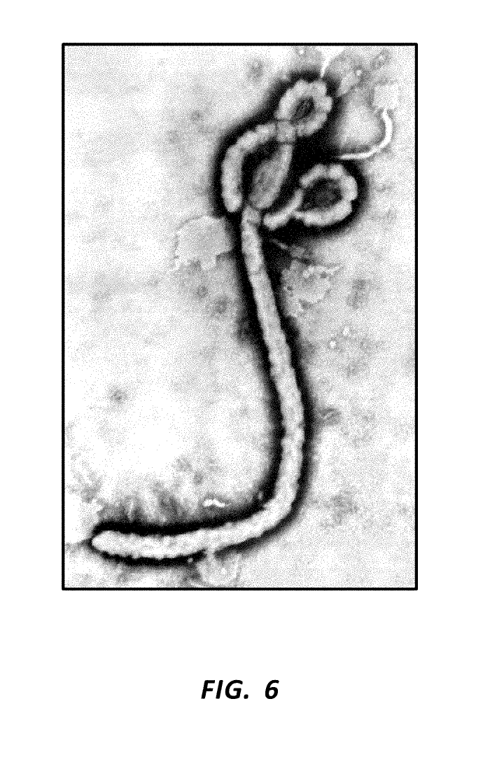

Notwithstanding the lethal nature of nonionic metal nanoparticles relative to microbes, they can be relatively harmless to humans, mammals, and healthy mammalian cells, which contain much more complex protein structures compared to simple microbes in which most or all vital disulfide bonds are shielded by other, more stable regions of the protein. FIG. 5 schematically illustrates a mammalian protein 810 with disulfide (S—S) bonds that are shielded so as to resist being catalytically denatured by an adjacent spherical-shaped nanoparticle 804. In many cases the nonionic nanoparticles do not interact with or attach to human or mammalian cells, remain in and follow fluid flow, do not cross barriers, remain in the vascular system, and can be quickly and safely expelled through the urine without damaging kidneys or other cells.

In the particular case of silver (Ag) nanoparticles, the interaction of the silver (Ag) nanoparticle(s) within a microbe has been demonstrated to be particularly lethal without the need to rely on the production of silver ions (Ag⁺) to provide the desired antimicrobial effects, as is typically the case with conventional colloidal silver compositions. The ability of silver (Ag) nanoparticles to provide effective microbial control without any significant release of toxic silver ions (Ag⁺) into the surrounding environment is a substantial advancement in the art. In addition, obtaining very small nanoparticles as described herein made of a mixture or alloy of gold and silver atoms, wherein the gold has greater bonding ability and silver greater anti-microbial activity, is a substantial advancement in the art.

As discussed herein, the size of the nanoparticles can be selected to target and selectively kill specific types of microbes, including Ebola viruses. By way of further example, sub-micron sized metal nanoparticles (e.g., gold or gold-silver alloy nanoparticles having a diameter of about 0.4 nm) may be most effective in killing Ebola viruses, which, as illustrated in FIG. 6, have an oddly elongated and looped configuration that can potential shield vital proteins from larger nanoparticles. Using very small nanoparticles as described herein, particularly nonionic metal nanoparticles, permits the nanoparticles to more easily penetrate into and promote denaturing reactions within the protein coat, such as cleavage of disulfide (S—S) bonds in capsid proteins of the virus. Additionally, or alternatively, such nanoparticles can also bind to glycoproteins of the protein coat. For example, nanoparticles having a size of about 2 nm to about 10 nm, or about 4 nm to about 10 nm, or about 6 nm to about 10 nm can bind and/or cleave the 3CSY glycoprotein located on the surface of the Ebola virus. Either or both mechanisms can prevent a virus from attaching to a host cell and introducing its genetic material therein. An Ebola virus that is rendered unable to attach to a host cell and/or inject genetic material into the host cell is essentially inactive and no longer pathogenic.

### Microbe-Specific Nanoparticles

### Anti-Viral Nanoparticles

In some embodiments, spherical-shaped nanoparticles designed to selectively and preferentially deactivate viruses can have a diameter of about 8 nm or less, or about 1 nm to about 7 nm, or about 2 nm to about 6.5 nm, or about 3 nm to about 6 nm. Within these size ranges it is possible to select “designer anti-viral particles” of specific size that are particularly effective in targeting a specific virus.

In some embodiments, spherical-shaped nanoparticles designed to selectively and preferentially deactivate viruses (e.g., viruses having an elongated and/or looped structure such as Ebola viruses) can have a particle size in a range of about 0.22 nm to about 2 nm, or about 0.22 nm to about 1.5 nm, or about 0.24 nm to about 1 nm, or about 0.27 nm to about 0.7 nm, or about 0.3 nm to about 0.5 nm, or about 0.35 nm to about 0.45 nm. Nanoparticles within these narrow size ranges can provide “designer anti-viral particles” that are particularly effective in targeting Ebola viruses.

In some embodiments, spherical-shaped nanoparticles designed to selectively and preferentially deactivate viruses having a surface 3CSY glycoprotein or similarly structured surface glycoprotein (e.g., Ebola viruses) can have a particle size in a range of about 2 nm to about 10 nm, or about 4 nm to about 10 nm, or about 6 nm to about 10 nm.

### Anti-Bacterial Nanoparticles

In some embodiments, spherical-shaped nanoparticles designed to selectively and preferentially deactivate bacteria can have a diameter of about 3 nm to about 14 nm, or about 5 nm to about 13 nm, or about 7 nm to about 12 nm, or about 8 nm to about 10 nm. Within these size ranges it is possible to select “designer anti-bacterial particles” of specific size that are particularly effective in targeting a specific bacterium.

### Anti-Fungal Nanoparticles

In some embodiments, spherical-shaped nanoparticles designed to selectively and preferentially deactivate fungi can have a diameter of about 9 nm to about 20 nm, or about 10 nm to about 18 nm, or about 11 nm to about 16 nm, or about 12 nm to about 15 nm. Within these size ranges it is possible to select “designer anti-fungal particles” of specific size that are particularly effective in targeting a specific fungus.

According to some embodiments, the antimicrobial metal nanoparticles will comprise at least one of silver or gold. In the case of larger metal nanoparticles for use in killing bacteria and fungi, the metal nanoparticles may primarily or exclusively comprise silver. However, in the case of smaller metal nanoparticles for use in killing viruses, the metal nanoparticles may primarily or exclusively comprise gold. Due to the nature of silver and gold atoms making up the nanoparticles, it has been found that gold nanoparticles are better able to hold together at very small sizes (e.g., smaller than about 5-7 nm) compared to silver nanoparticles. On the other hand, a gold-silver alloy provides the particle stabilizing activity of gold and the higher microbe killing activity of silver.

As discussed herein, the size of the nanoparticles can be selected to target and selectively kill specific types of microbes. By way of further example, sub-micron sized metal nanoparticles (e.g., gold nanoparticles having a diameter of about 0.4 nm) may be most effective in killing Ebola viruses, which have an oddly elongated and looped configuration that can potential shield vital proteins from larger nanoparticles. By way of further example, in the case of treating citrus greening disease, the nanoparticles can have a particle size in a range of about 1 nm to about 25 nm, or about 2 nm to about 15 nm, or about 2 nm to about 7 nm, or about 3 nm to about 6 nm.

### Multi-Component Nanoparticle Compositions

In some embodiments, coral-shaped metal nanoparticles can be used in conjunction with spherical-shaped metal nanoparticles. In general, spherical-shaped metal nanoparticles can be smaller than coral-shaped metal nanoparticles and in this way can provide very high surface area for catalyzing desired reactions or providing other desired benefits. On the other hand, the generally larger coral-shaped nanoparticles can exhibit higher surface area per unit mass compared to spherical-shaped nanoparticles because coral-shaped nanoparticles have internal spaces and surfaces rather than a solid core and only an external surface. In some cases, providing antimicrobial compositions containing both spherical-shaped and coral-shaped nanoparticles can provide synergistic results. For example, coral-shaped nanoparticles can help carry and/or potentiate the activity of spherical-shaped nanoparticles in addition to providing their own unique benefits.

In some embodiments, the antimicrobial compositions may include both spherical-shaped and coral-shaped nanoparticles. In some embodiments, the mass ratio of spherical-shaped nanoparticles to coral-shaped nanoparticles in the nanoparticle composition can be in a range of about 1:1 to about 50:1, or about 2.5:1 to about 25:1, or about 5:1 to about 20:1, or about 7.5:1 to about 15:1, or about 9:1 to about 11:1, or about 10:1. The particle number ratio of spherical-shaped nanoparticles to coral-shaped nanoparticles in the nanoparticle composition can be in a range of about 10:1 to about 500:1, or about 25:1 to about 250:1, or about 50:1 to about 200:1, or about 75:1 to about 150:1, or about 90:1 to about 110:1, or about 100:1.

### Carriers

In some embodiments, a nanoparticle composition includes a carrier for delivering the metal nanoparticles onto or into a living or non-living substrate. The carrier can be a liquid, gel, or solid. Some carriers may be more suitable than others depending on the living or non-living substrate being treated. For example, the solubility characteristics of the carrier can be selected to maximize or otherwise provide a desired diffusion throughout a treated organism part and/or another portion of the organism in contact with the treated organism part.

Examples of compounds that can be utilized for topical applications and can be used as carriers include, but are not limited to, water, alcohols, ketones, esters, citrus oils, essential oils, vegetable and other plant and natural oils, triglycerides, ethers, organic solvents, methanol, ethanol, isopropyl alcohol, other alcohols, glycols, glycerin, polyols, 1,3-propandiol, petroleum jelly, waxes, polymers, polymerizable materials, and surfactants.

In one embodiment, the carrier is a cream or lotion including a glycerin and/or stearic acid cream base optionally containing oils such as coconut oil, olive oil, grape seed oil, shea butter, mango butter, and/or vitamin E oil along with an emulsifying wax.

In other embodiments the carrier is a water or combined water and alcohol solution which itself contains a micro to millimolar concentration of a separate stabilizing agent dissolved into the carrier so as to maintain the nanoparticles within the overall composition.

Exemplary carriers for nasal or pulmonary aerosol or inhalation administration include solutions in saline which can contain, for example, benzyl alcohol or other suitable preservatives, absorption promoters to enhance bioavailability, and/or other solubilizing or wetting or dispersing agents, such as glycerin, a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycethanol), a condensation product of ethylene oxide with a partial ester derived from a fatty acid and a hexitol anhydride (e.g., polyoxyethylene sorbitan monooleate); polysaccharides and polysaccharide-like compounds (e.g. dextran sulfate); and glycoaminoglycans and glycosaminoglycan-like compounds (e.g., hyaluronic acid), for example. In some embodiments, the nanoparticles and additional stabilizing agents and/or carriers are formulated as dry powders (e.g., powders useful for administering with dry powder inhalers).

Exemplary aerosols useful for nasal and/or inhalation administration include a vaporizable propellant, such as low molecular weight hydrofluorocarbons or hydrocarbons that are liquid when constrained in a suitable container and are biocompatible and non-irritating. Ingredients such as water, alcohol, propylene glycol, and polyethylene glycols can be additionally included. Other embodiments, also useful for nasal and/or inhalation administration, are provided as sprays (e.g., omitting an aerosol propellant). Such spray formulation may be provided as a solution, suspension, or emulsion capable of forming a fine mist for administration, and in some embodiments, may include saline and/or be isotonic.

Exemplary injectable solutions include an aqueous emulsion or oleaginous suspension or saline solution (e.g., isotonic, hypotonic, or hypertonic, optionally including dextrose and/or other electrolytes or additives). Such compositions can also include suitable dispersing or wetting agents. The sterile injectable preparation may also be formed in a non-toxic parenterally acceptable diluent or solvent, such as a solution in 1,2-propanediol (propylene glycol). Additional examples include solutions or suspensions which can contain, for example, suitable non-toxic diluents or solvents, such as mannitol, 1,3-butanediol, water, Ringer's solution, an isotonic sodium chloride solution, or other suitable dispersing or wetting and suspending agents, including synthetic mono- or diglycerides, and fatty acids, including oleic acid, or Cremaphor.

Gels known in the art can be used as carriers, such as gels containing one or more of the foregoing liquid components together with known gelling agents. Gel compositions can more easily adhere to a living or non-living substrate being treated. An exemplary gel carrier can include mineral oil gelled with polyethylene.

Solid carriers can be used for different reasons, such as to elute nanoparticles into an organism over time. Examples of solid carriers include, but are not limited to, polymers, rubbers, elastomers, foams, and gums. Depending on the characteristics of the organism to be treated and the desired rate of elution, one of skill in the art can select an appropriate solid carrier material.

In some embodiment, an antimicrobial composition can be formulated so that the metal nanoparticles are included in a concentration so that a measured quantity of the nanoparticle composition, when applied onto or into an organism or organism part, will provide a predetermined concentration or quantity of metal nanoparticles. The nanoparticle composition can have a higher concentration of nanoparticles that become diluted when mixed with other liquids applied to or naturally contained within the organism or organism part. Depending on the organism or organism part being treated, the nature of the nanoparticles being added, and the type of carrier being used, the antimicrobial composition may contain about 10 ppb (parts per billion) to about 100 ppm (parts per million) by weight of the antimicrobial composition, or about 15 ppb to about 90 ppm, or about 100 ppb to about 75 ppm, or about 500 ppb to about 60 ppm of metal nanoparticles by weight, or about 1 ppm to about 50 ppm, or about 2 ppm to about 25 ppm, or about 3 ppm to about 20 ppm metal nanoparticles by weight of the antimicrobial composition.

In some embodiments, the antimicrobial composition can also include one or more optional components or adjuvents to provide desired properties, including, but not limited to food, vitamins, minerals, antimicrobial agents, electrolytes, moisturizers, emollients, antiseptics, and/or plant extracts.

In some embodiments, the carrier may also function as, or may include, a stabilizing agent. For example, in some embodiments it may be desirable to have different specifically sized nanoparticles within the same solution to take advantage of each of the different properties and effects of the different particles. However, when differently sized particles are mixed into a single solution, the overall long-term stability of these particles within that single solution may be substantially diminished as a result of unequal forces exerted on the various particles causing eventual agglomeration of the particles. This phenomenon may become even more pronounced when that solution is either heated or cooled significantly above or below standard room temperature conditions.

Examples of stabilizing agents include alcohols (e.g., ethanol, propanol, butanol, etc.), polyphenols (e.g., arjuna bark extract, grape seed extract, etc.), mono-glycerides, di-glycerides, or triglycerides (e.g., grape seed oil, coconut oil, and the like), oils (e.g., lavender), other terpenes, amine compounds (e.g., mono-, di-, or tri-ethanol amine), carbohydrates (e.g., sucrose, fructose), liposomes, creams, other emulsions, and other polymers.

In some embodiments, stabilizing agents are dissolved within a separate carrier in the micro- to milli-molar concentration range with the upper range limitation typically being constrained not by efficacy but by product cost.

These various stabilizing agents have the capacity to hold the at least two differently sized and/or shaped nanoparticles in suspension and deliver these nanoparticles into the treatment area of a person or animal without so powerfully retaining the nanoparticles so as to diminish the antimicrobial properties of the nanoparticles.

### Treatment Methods

In some embodiments, a method of method of killing or deactivating a microbe comprises: (1) applying an antimicrobial composition comprising a carrier and metal nanoparticles onto or into a substrate containing microbes; and (2) the antimicrobial composition killing or denaturing the microbes. The substrate can be a living organism, such as an organism that is infected with, or may become infected with, a disease caused by a microbe. Alternatively, the substrate can be a non-living object that has come into contact, or is at risk of coming into contact, with a disease causing or otherwise unwanted microbe, such as clothing, bedding, wound dressings, medical devices or implants, food, intravenously injectable liquids, farm equipment, animal feeding devices, watering troughs, and the like.

In some embodiments, metal nanoparticles kill bacteria or fungi by entering the cell and catalyzing protein denaturing reactions. In some embodiments, metal nanoparticles “deactivate” viruses by attaching to glycoproteins and/or catalyzing protein denaturing reactions in the protein coat. Examples of protein denaturing reactions include reactions involving one or more of disulfide (S—S) bond cleavage and formation of active oxygen species that attack amide or other bonds.

In some embodiments, a method of deactivating a virus comprises: (1) applying an anti-viral composition comprising metal nanoparticles having a selected particle size onto or into a substrate containing a virus, and (2) the anti-viral composition deactivating the virus.

In some embodiments, a method of deactivating Ebola viruses comprises: (1) applying an anti-viral composition comprising a carrier and metal nanoparticles onto or into an animal or non-living substrate contaminated with Ebola viruses; and (2) the metal nanoparticles deactivating Ebola viruses by attaching to glycoproteins and/or denaturing one or more proteins in the Ebola viruses. The substrate can be a living organism, such as an organism that is infected with, or may become infected with, Ebola viruses. Alternatively, the substrate can be a non-living object that can come into contact with an organism and potentially cause or spread disease, such as clothing, beading, wound dressing, medical device or implant, food, IV liquid, farm equipment, animal feeding devices, watering troughs, and the like.

In some embodiments, metal nanoparticles deactivate viruses by attaching to glycoproteins and/or catalyzing protein denaturing reactions in the protein coat. Examples of protein denaturing reactions include reactions involving one or more of disulfide (S—S) bond cleavage and formation of active oxygen species that attack amide or other bonds.

In some embodiments, the method may comprise administering a pharmaceutically acceptable quantity of the anti-viral composition to a mammal in order to treat or prevent Ebola virus disease. The anti-viral composition can be administered in any appropriate fashion, including but not limited to, intravenously, via inhalation, orally, and/or topically. Advantageously, the metal nanoparticles selectively deactivate Ebola viruses without harming humans or animals contacting the metal nanoparticles.

In some embodiments, the method may comprise applying the anti-viral composition to one or more of medical equipment, clothing, bandages, waste receptacles, syringes, syringe needles, inhalation equipment, or implants.

In some embodiments, a method of treating or preventing Ebola virus disease comprises: (1) administering a pharmaceutically acceptable quantity of spherical-shaped nonionic metal nanoparticles having a particle size in a range of about 0.22 nm to about 2 nm to a living organism, and (2) the anti-viral composition deactivating the virus.

In some embodiments, a method of killing a bacterium comprises: (1) applying an anti-bacterial composition comprising metal nanoparticles having a selected particle size onto or into a substrate containing a bacterium, and (2) the anti-bacterial composition killing the bacterium.

In some embodiments, a method of killing a fungus comprises: (1) applying an anti-fungal composition comprising metal nanoparticles having a selected particle size onto or into a substrate containing a fungus, and (2) the anti-bacterial composition killing the fungus.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A method of selectively killing or deactivating a target microbe, comprising:
applying an antimicrobial composition to the target microbe or to a substrate containing the target microbe, the antimicrobial composition comprising:
a carrier; and
a plurality of spherical-shaped, nonionic metal nanoparticles formed by laser ablation having a mean diameter in a range of 1-20 nm so as to selectively kill or deactivate the target microbe, wherein at least 99% of the spherical metal nanoparticles have a diameter within ±3 nm of the mean diameter; and
the antimicrobial composition selectively killing or deactivating the target microbe, which is selected from a bacterium, a virus, or a fungus, and wherein:
when the microbe is a bacterium the metal nanoparticles have a mean diameter in a range of 3 nm to 14 nm to selectively kill the bacterium,
when the microbe is a virus the metal nanoparticles have a mean diameter in a range of 1 nm to 7 nm to selectively kill the virus, or
when the microbe is a fungus the metal nanoparticles have a mean diameter in a range of 9 nm to 20 nm to selectively kill the fungus.

2. The method of claim 1, wherein the substrate is a non-living object.

3. The method of claim 1, wherein the substrate is a living organism.

4. The method of claim 1, wherein the microbe is a bacterium and the metal nanoparticles have a mean diameter in a range of 5 nm to 13 nm to selectively kill the bacterium.

5. The method of claim 1, wherein the microbe is a bacterium and the metal nanoparticles have a mean diameter in a range of 7 nm to 12 nm to selectively kill the bacterium.

6. The method of claim 1, wherein the microbe is a bacterium and the metal nanoparticles have a mean diameter in a range of 8 nm to 10 nm to selectively kill the bacterium.

7. The method of claim 1, wherein the microbe is a virus and the metal nanoparticles have a mean diameter in a range of 2 nm to 6.5 nm to selectively kill the virus.

8. The method of claim 1, wherein the microbe is a virus and the metal nanoparticles have a mean diameter in a range of 3 nm to 6 nm to selectively kill the virus.

9. The method of claim 1, wherein the microbe is a fungus and the metal nanoparticles have a mean diameter in a range of 10 nm to 18 nm to selectively kill the fungus.

10. The method of claim 1, wherein the microbe is a fungus and the metal nanoparticles have a mean diameter in a range of 11 nm to 16 nm to selectively kill the fungus.

11. The method of claim 1, wherein the microbe is a fungus and the metal nanoparticles have a mean diameter in a range of 12 nm to 15 nm to selectively kill the fungus.

12. The method of claim 1, wherein at least 99% of the spherical metal nanoparticles have a diameter within ±1 nm of the mean diameter.

13. The method of claim 1, wherein the antimicrobial composition further comprises coral-shaped metal nanoparticles, each coral-shaped metal nanoparticle having a non-uniform cross section and a globular structure formed by multiple, non-linear strands joined together without right angles.

14. The method of claim 1, wherein the carrier is a liquid in which the metal nanoparticles are colloidally dispersed.

15. The method of claim 1, wherein the metal nanoparticles have a concentration in a range of about 10 ppb to about 100 ppm by weight of the antimicrobial composition.

16. The method of claim 1, wherein the metal nanoparticles have a concentration in a range of about 100 ppb to about 75 ppm by weight of the antimicrobial composition.

17. The method of claim 1, wherein the metal nanoparticles have a concentration in a range of about 1 ppm to about 50 ppm by weight of the antimicrobial composition.

18. The method of claim 1, wherein the spherical metal nanoparticles comprise silver, gold, or an alloy of silver and gold.

19. A method of selectively killing or deactivating a bacterium, comprising:
applying an antimicrobial composition to the bacterium or to a substrate containing the bacterium, the antimicrobial composition comprising:
a carrier; and
a plurality of spherical-shaped, nonionic metal nanoparticles formed from silver or a silver alloy and having a mean particle size in a range of 3-14 nm to selectively kill or deactivate the bacterium, wherein at least 99% of the spherical metal nanoparticles have a particle size within ±3 nm of the mean particle size; and
the antimicrobial composition selectively killing or deactivating the bacterium without release of silver ions.

20. A method of selectively killing or deactivating a virus, comprising:
applying an antimicrobial composition to the virus or to a substrate containing the virus, the antimicrobial composition comprising:
a carrier; and
a plurality of spherical-shaped, nonionic metal nanoparticles formed from silver or a silver alloy and having a mean particle size in a range of 1-7 nm to selectively kill or deactivate the virus, wherein at least 99% of the spherical metal nanoparticles have a particle size within ±1 nm of the mean particle size; and
the antimicrobial composition selectively killing or deactivating the virus without release of silver ions.
